SEQUENCE LISTING (1 sequence)

SEQ ID NO: 1 - DNA, 1261 bp, Homo sapiens
  misc_feature (298)..(1095): IRES Sequence
  misc_feature (298)..(656): IRES Module 1
  misc_feature (656)..(1095): IRES Module 2 <s300> Jimenez J., An internal ribosome entry site mediates translation of lymphoid enhancer factor-1 RNA 11 9 1385-1399 2005-09
tcagcgccggcgaggcgcgggaggaggagaagcagtggggaggcgcagccgctcacctgcggggcagggcgcggaggagggacccgggctgcgcgctctcgggccgaggaaccaggacgcgcccggagcctcgcacgcggccaagctcggggcgtcccctcccctcggccgggcgaactcaaggggcgcagctctttgctttgacagagctggccggcggaggcgtgcagagcggcgagccggcgagccaggctgagaaactcgagccgggaacaaagaggggtcggactgagtgtgtgtgtcggctcgagctccgggcagaggcatttgggcccgaggcccccgctgtgactccccgagactccgcagtgccctccactgcggagtccccgcgcttgccggcaaaaactttattcttggcaaacttctctttctcttcccctcctcctcggcccccatcttctgctcctcctccttctctagcagattaaatgagcctcgagaagaaaaaccgaagcgaaagggaagaaaataagaagatctaaaacggacatctccagcgtgggtggctcctttttctttttctttttttcccacccttcaggaagtggacgtttcgttatcttctgatccttgcaccttcttttggggcaaacggggcccttctgcccagatcccctctcttttctcggaaaacaaactactaagtcggcatccggggtaactacagtggagagggtttccgcggagacgcgccgccggaccctcctctgcactttggggaggcgtgctccctccagaaccggcgttctccgcgcgcaaatcccggcgacgcggggtcgcggggtggccgccggggcagcctcgtctagcgcgcgccgcgcagacgcccccggagtcgccagctaccgcagccctcgccgcccagtgcccttcggcctcgggggcgggcgcctgcgtcggtctccgcgaagcgggaaagcgcggcggccgccgggattcgggcgccgcggcagctgctccggctgccggccggcggccccgcgctcgcccgccccgcttccgcccgctgtcctgctgcacgaacccttccaactctcctttcctcccccacccttgagttacccctctgtctttcctgctgttgcgcgggtgctcccacagcggagcggagattacagagccgccgggatgccccaactctccggaggaggtggcggcggcgggggggacccggaactctgcgccacggacgagatga

(19) United States
(12) Patent Application Publication
(10) Pub. No.: US 2008/0096228 A1
(43) Pub. Date: Apr. 24, 2008

(54) Compositions And Methods Relating To Mammalian Internal Ribosome Entry Sites

(75) Inventors: Marian L. Waterman, Irvine, CA (US); Judith Jimenez, Irvine, CA (US); S. Tiong Ong, Irvine, CA (US)

Correspondence Address: FISH & ASSOCIATES, PC;ROBERT D. FISH, 2603 Main Street, Suite 1050, Irvine, CA, 92614-6232, US

(73) Assignee: THE REGENTS OF THE UNIVERSITY OF CALIFORNIA, Oakland, CA (US)

(21) Appl. No.: 11/835,974

(22) Filed: Aug. 8, 2007

Related U.S. Application Data
(60) Provisional application No. 60/836,492, filed on Aug. 8, 2006.

(51) Int. Cl. G01N 33/53; C07H 21/00; G01N 33/573; C12N 5/06

(52) U.S. Cl. 435/7.4; 435/325; 435/372; 435/7.1; 536/23.2; 536/23.5

(57) ABSTRACT

Contemplated compositions and methods are directed to IRES sequences and their use, especially in the context of CML. Among other contemplated aspects IRES sequences described herein are highly active in recombinant in vitro systems and allow high-level of recombinant protein from two, typically opposite reading frames. In vivo, and particularly in lymphocytes of CML patients, expression of genes associated with the IRES (e.g., LEF-1) is at least partially controlled by Bcr-Abl activity, which may be used as a diagnostic tool for CML patients.

This application claims priority to our copending U.S. provisional patent application with the Ser. No. 60/836,492, which was filed Aug. 8, 2006.

This invention was made with Government support under Grant Nos. CA083982 and GM067285, awarded by the National Institutes of Health. The Government has certain rights in this invention.

### FIELD OF THE INVENTION

The field of the invention is nucleic acids and methods of use thereof, especially as they relate to internal ribosomal entry sites and their use for dicistronic gene expression, or prognosis, diagnosis, and/or treatment of drug resistance in chronic myelogenous leukemia.

### BACKGROUND OF THE INVENTION

Chronic myeloid leukemia (CML) is a malignant stem cell disorder that has served as a model for other cancers due to its multi-step evolution with defined stages ranging from chronic phase and accelerated phase to blast phase. During the chronic phase, a reciprocal chromosomal translocation between the long arms of chromosomes 9 and 22 [t(9;22)(q34;q11)], known as the Philadelphia chromosome, is commonly formed. This translocation generates the fusion protein Bcr-Abl, which is a constitutively active tyrosine kinase shown to induce leukemia in mice when transfected into marrow cells. The demonstration of the pathogenic relevance of the Bcr-Abl fusion protein led to the search for inhibitors such as imatinib mesylate (imatinib), a drug that binds the ATP-binding site of the fusion protein and stabilizes the kinase in its catalytically inactive conformation. Imatinib therapy has proven to be well-tolerated and effective in chronic phase patients. However, some patients develop resistance via either mutations of the ATP-binding site (more than 30 different mutations have been recognized) and/or gene amplification.

Lymphoid Enhancer Factor-1 (LEF-1), a transcription factor that mediates Wnt signals via interaction with beta-catenin, is often expressed in cancers derived from aberrant Wnt signaling. Recently, it has been reported that LEF1 expression is elevated in CML. While normal granulocyte-macrophage progenitors typically have reduced expression of beta-catenin or its transcriptional co-activator, LEF-1 (as compared with normal hematopoietic stem cells), CML granulocyte-macrophage progenitors from patients in the blast phase express increased levels of both transcripts, which most likely contribute to the nuclear accumulation of beta-catenin. More recently, the inventors discovered that LEF-1 translation is mediated by an internal ribosomal entry site (IRES).

Internal ribosomal entry sites are known to mediate and/or enhance dicistronic expression in various organisms, and are most notably associated with viral gene expression. Among other known internal ribosomal entry sites, various exemplary entry sites are described for murine retrotransposons (e.g., U.S. Pat. No. 5,925,565), certain type C retroviruses (e.g., U.S. Pat. No. 6,783,977), selected drosophila genes (U.S. Pat. App. No. 2004/0082034), enteroviruses (U.S. Pat. App. No. 2005/0112095), errantiviruses (e.g., WO02/22839), cardiovirus 2A (U.S. Pat. App. No. 2005/0019808 and U.S. Pat. No. 4,937,190), and plant viruses (U.S. Pat. App. No. 2004/0055037).

Further known IRES sequences may also be entirely synthetic and were described as adenine rich sequences with 40-100 mol-% of adenine in the regulatory domain as taught in U.S. Pat. App. No. 2005/0059004. Other synthetic sequences with IRES activity were reported in U.S. Pat. App. No. 2004/0043468. While most of these known sequences allow dicistronic expression of genes in a cell, various disadvantages nevertheless remain. Most significantly, levels of expression driven from the IRES are relatively low. Furthermore, expression in most of these systems is mediated by RNA injected or otherwise provided to the cytoplasm, which has its own challenges. To overcome at least some of the disadvantages associated with RNA handling or other manipulations, certain IRES expression vectors have been developed. However, all or almost all of such vectors still lack the desirable high expression rates.

Therefore, while some therapeutic and diagnostic approaches for detection and treatment of CML are known in the art, all or almost all of them suffer from one or more disadvantages. Moreover, LEF-1 related IRES sequences per se and up-regulated expression of sequences under their control in certain neoplastic diseases are not well understood. Consequently, there is still a need to provide improved composition and methods relating to LEF-1 related IRES sequences, especially as it relates to CML.

### SUMMARY OF THE INVENTION

The present invention is directed to compositions and methods that are related to internal ribosome entry site sequences, and especially to diagnostic methods and recombinant constructs comprising same. Most preferably, the IRES is a mammalian IRES and has a sequence according to SEQ ID NO:1 or hybridizes under stringent conditions to same, and diagnostic methods are drawn to patients suffering from CML.

In one aspect of the inventive subject matter, a method of diagnosing a patient suffering from leukemia includes a step of collecting leukocytes from the patient. After optional culture of the leukocytes, a first expression level of LEF-1 protein is measured in the leukocytes prior to administration of a drug, and a second expression level of the LEF-1 protein is measured in the leukocytes after administration of the drug. In yet another step, the first and second expression levels are compared, optionally against an expression level of LEF-1 in leukocytes of a healthy person, to obtain a test result, and a treatment option is determined using the test result.

In particularly preferred aspects, first and second expression levels are measured using a labeled antibody having specificity towards LEF-1 protein (e.g., in a Western blot), and where desirable, additional measurements may be taken. Most preferably, such methods include the further steps of (a) measuring kinase activity of Bcr-Abl in the leukocytes (e.g., using a fluorogenic or chromogenic substrate, optionally in Western blot format) prior to administration of the drug, (b) measuring kinase activity of Bcr-Abl in the leukocytes after administration of the drug to obtain a second test result, and further using the second test result in the step of determining the treatment option.

Typically, and particularly where the leukemia is CML, preferred drugs include Bcr-Abl kinase inhibitor, and most typically Dasatinib, Imatinib, and/or Nilotinib. Where the expression level of LEF-1 is reduced after administration of the drug, and optionally where the Bcr-Abl kinase activity is reduced after administration, the treatment option will comprise administration of a Bcr-Abl inhibitor. On the other hand, and especially where a patient is already on a therapy that includes administration of a Bcr-Abl inhibitor and the LEF-1 expression level and/or Bcr-Abl activity is not significantly reduced, the treatment option may comprise administration of a different Bcr-Abl inhibitor or alternative therapy directed against alternative targets in CML.

In another aspect of the inventive subject matter, an isolated IRES sequence, optionally upstream and/or downstream of an open reading frame coding for an expression product, is contemplated, wherein the IRES has a sequence that allows (a) hybridization under stringent conditions to the IRES of SEQ ID NO:1, and (b) increased expression of a reference peptide in an amount of at least 5 times relative to expression of the reference peptide under control of a mammalian Kv1.4 IRES in the same expression host.

Preferably, the IRES sequence has at least one of 70% (more preferably at least 80%, and most preferably at least 95%) sequence homology and 60% (more preferably at least 70%, and most preferably at least 90%) sequence length of the IRES of SEQ ID NO:1, and the IRES sequence provides an at least 10 times (more preferably at least 20%, and most preferably at least 60%) increased expression of the reference peptide. Such sequences may further include distinct upstream and downstream ORFs for expression in the host cell (e.g., luciferase, fluorescent proteins, etc.), sequences effective to allow at least one of integration of the sequence into a host genome and replication in the host cell, and/or selectable markers. Consequently, cells and especially leukocytes comprising such recombinant IRES sequences are also contemplated.

Various objects, features, aspects and advantages of the present invention will become more apparent from the following detailed description of preferred embodiments of the invention, along with the accompanying drawing.

### BRIEF DESCRIPTION OF THE DRAWING

FIG. 1 depicts Western blots of LEF-1 expression in primary CML from various patients in chronic and blast phases.

FIGS. 2A and 2B depict Western blots showing effects of imatinib on LEF-1 expression in K562 cells.

FIGS. 3A and 3B depict Western blots showing LEF-1 expression in selected Bcr-Abl expressing cells

FIG. 4A schematically illustrates selected vector constructs, and FIGS. 4B-4D depict graphs representing effects of imatinib, rapamycin or both on LEF-1 IRES activity in Ba/F3-Bcr-Abl-WT cells (4B), Ba/F3-Bcr-Abl-T3151 cells (4D), and K562 cells (4D) transfected with the dicistronic constructs.

FIGS. 5A-5C depict graphs representing effects of imatinib on luciferase activities in cell lysates by the dicistronic mRNAs representing cap-dependent translation (5A), luciferase activity generated by the LEF-1 (5B), and luciferase activity generated by CVB3 IRES.

FIG. 6 is a schematic model of Wnt Signaling and synergistic Bcr-Abl upregulation of LEF-1 expression in CML.

### DETAILED DESCRIPTION

The inventors have discovered a mammalian (here: human) IRES that allows dicistronic expression of flanking genes in significant quantities. Moreover, such IRES elements exhibit the unexpected characteristic of even higher expression rates where the IRES sequence is present in the nucleus of a eukaryotic cell at least for some time. Still further, it is contemplated that IRES sequences presented herein may not only be used as a tool for recombinant production of a gene of interest, but that such IRES sequences may also be employed in diagnostic or even therapeutic context for various diseases, and particularly CML.

### Contemplated Sequences, Vectors, Cells and Uses

In one preferred aspect of the inventive subject matter the IRES sequence has a sequence as SEQ ID NO:1 in the sequence listing below, starting at about nucleotide 298 and ending at about nucleotide 1095. The IRES may further be characterized as having at least two modules, with module 1 starting at about nucleotide 298 and ending at about nucleotide 656, and module 2, starting at about nucleotide 656 ending at about nucleotide 1095. Such IRES sequence and its variations with IRES activity may be isolated and incorporated in any nucleic acid construct, and most preferably into a DNA vector having dicistronic arrangement. In one exemplary aspect, genes expressed in cells transformed with such a construct exhibit an unusually strong expression as compared to other known IRES expression constructs. Moreover, the inventors also discovered that the dicistronic expression may be further increased by having the IRES sequence in the nucleus for at least some time (e.g., where the IRES sequence is transcribed from a DNA located in the nucleus).

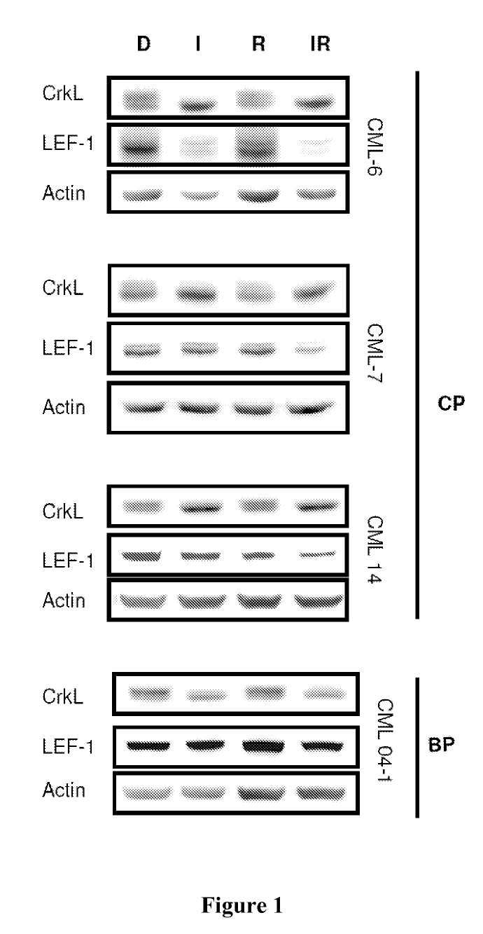

However, in alternative aspects of the inventive subject matter, it should be recognized that the IRES sequence need not be limited to the hLEF sequence as depicted in FIG. 1 above, but may also be modified to some extent. It should be appreciated that as with previously known IRES sequences, the enhancement of translation is not strictly limited to a specific nucleotide sequence, but that the IRES sequence is tolerant to numerous modifications. Therefore, it is also contemplated that all sequences are suitable that have at least 70%, more preferably at least 85%, and most preferably at least 95% sequence homology and/or at least 60%, more preferably at least 80%, and most preferably at least 90% of the full length IRES as hLEF depicted in FIG. 1. Hybridization also characterized as hybridizing under stringent conditions (e.g., hybridization in 4×SSC at 62-67° C., followed by washing in 0.1×SSC at 62-67° C. for approximately an hour. Alternatively, stringent hybridization conditions can be performed in 45-55% formamide, 4×SSC at 40-45° C. [See, T. Maniatis et. al., Molecular Cloning (A Laboratory Manual); Cold Spring Harbor Laboratory (1982), pages 387 to 389].)

Depending on the degree of truncation, it is generally contemplated that the modified IRES sequence provides an at least 5 times, more typically at least 20 times, and most typically at least 60 times increased expression of a reference peptide as compared to expression of the reference peptide under the control of a commonly used mammalian IRES (e.g., Kv 1.4 IRES).

Thus, suitable sequence modifications may include deletions, transitions, transversions, insertions, inversions, and even replacement of a nucleotide with a synthetic nucleotide analog. Such modifications may be made based on a particular rationale (e.g., site-directed to eliminate or add a start codon), or may be random (e.g., using error prone PCR to generate a library of variants). Furthermore, it should be recognized that sequence variability may be achieved by isolating hLEF IRES equivalent sequences from mammals (or other animals). For example, alternative sources include rat, cow, mouse, dog, etc. On the other hand, and especially where expression is to be still further increased, it is contemplated that a recombinant construct may comprise two or more copies of the IRES contemplated herein, wherein each of the copies may or may not be identical.

It should further be appreciated that the sequence context of recombinant IRES sequences contemplated herein may vary considerably, and it is noted that all reasonable constructs are contemplated herein. For example, in especially preferred aspects, a dicistronic vector may be constructed in which contemplated IRES is flanked by two multiple cloning sites, wherein such vector will further include at least one of a bacterial origin of replication (e.g., oriR1), selectable marker(s) for bacteria and/or eukaryotic cells (e.g., bla^{R}/neo^{R}), promoter elements, enhancers, LTRs, and optionally a reference gene (e.g., luciferase, green fluorescent protein, etc.) under the control of the IRES. Such recombinant nucleic acids may be present as double stranded DNA, which may be circular or linear, or as an RNA that may be isolated, or produced in vitro/vivo.

Consequently, the inventors also contemplate cells that may carry the recombinant IRES nucleic acid, wherein such cells may be transiently transfected or stably transfected. Where cells are transfected, it is generally preferred that the nucleic acid is a DNA that is located in the nucleus. For example, such transfection may be performed using transfection agents (lipofectin, liposomes, etc.), viral transfection, electroporation, etc. Thus, the DNA may be integrated into the genome of a cell or be present as an extrachromosomal unit (which may or may not replicate), and may be present in a primary cell, a cultured cell, an immortalized cell, a transgenic cell and/or in a stem cell. Further details, contemplations, and experimental results are provided in RNA, 2005 September; 11(9):1385-99, which is incorporated by reference herein.

### Contemplated Diagnostic Assays and Methods

While it is well known that LEF1 transcripts are up-regulated in the blast phase of CML, translation of these mRNAs has not been examined. Here, the inventors surprisingly discovered that LEF-1 is a target of Bcr-Abl since production of LEF1 mRNA and LEF-1 protein are down-regulated by the Bcr-Abl inhibitor, imatinib. Therefore, LEF-1 appears to be a target of Bcr-Abl signaling and may contribute to the increased survival and proliferation of myeloid progenitor populations. Based on these and other considerations, it is contemplated that LEF-1 plays a role in the pathogenesis of CML (as has previously been demonstrated by the capability of LEF-1 protein to transform cells, and as LEF-1 is uniformly expressed in all primary CML samples tested).

Furthermore, the degree to which LEF-1 translation/expression could be inhibited by imatinib in vitro correlated with clinical outcome, since it was found that a lack of inhibition was associated with the eventual development of clinical imatinib-resistance. These observations also indicate that LEF-1 expression may play a role in conferring drug resistance. Of the known mechanisms associated with clinical imatinib-resistance, Abl kinase domain mutations account for the majority of cases. However, recent work has shown that in myeloid blast phase, the proportion of patients without kinase domain mutations to account for their imatinib-resistance may be as high as 41%, suggesting that Bcr-Abl-independent mechanisms of resistance become increasingly important in the later phases of disease.

While examining LEF-1 protein production in cells expressing Bcr-Abl, the inventors observed that imatinib specifically down-regulated LEF-1 protein synthesis while rapamycin has a more modest effect. Although the inventors previously demonstrated that combined treatment with rapamycin and imatinib results in a significantly greater downregulation of cap-dependent translation than either agent alone, Western blot results show that rapamycin makes only a slight contribution, indicating that LEF-1 protein expression occurs via a different mechanism. On the other hand, the dicistronic reporter assay reveals a mild sensitivity to rapamycin (38% decrease), which may derive from the experimental system where a dicistronic mRNA is produced from a transiently transfected plasmid. Consequently, it is contemplated that imatinib must inhibit cap-independent mechanisms via an alternate mode that rapamycin does not target effectively.

While the RNA transfection reveals an imatinib-sensitive step in the cytoplasm, several post-transcriptional and translational mechanisms (e.g., RNA loading and nuclear export) might regulate LEF-1 expression in CML since the greatest effect of imatinib is observed in a DNA transfection, which requires the mRNA to pass through the nucleus. Based on the inventors' findings and other factors, the inventors contemplate that LEF-1 expression in CML is enhanced via Bcr-Abl's upregulation of nuclear RNA binding proteins, such as hnRNPs, as is the case for c-MYC. The imatinib-sensitivity also observed in the cytoplasm may be due to negative effects on the pool of shuttling RNA binding proteins (e.g., hnRNPs) in the cytoplasm that can still interact with LEF1 mRNA.

As can be taken from the experiments below and other data (not shown), Bcr-Abl is a positive regulator of Wnt Signaling at both the levels of transcription and translation. While the experimental data were mainly focused on LEF-1, it is generally contemplated that these effects, at least on translation, are global. That is, cap-independent translation is generally elevated in CML. The Bcr-Abl effect on LEF-1 transcription is less well defined, but likely to be the result of a general upregulation of transcription since GAPDH mRNA is also affected. Consequently, it is contemplated that Wnt Signal transduction in relatively late stages of CML results in increased transcription of target genes such as LEF1 and c-MYC. Bcr-Abl may then enhance translation of the two IRES-containing mRNAs at multiple steps: Bcr-Abl may modestly up-regulate transcription of LEF1 in addition to enhancing transcription of RNA binding factors that participate in either nuclear export and/or IRES-mediated translation, resulting in elevated LEF-1 protein expression. Thus, it should be appreciated that Bcr-Abl and Wnt signals may synergize to create a potent transformation signal that results in fast cycling, undifferentiated tumor cells.

As the inventors have discovered that IRES-dependent translation of human lymphoid enhancer factor 1 (hLEF-1) mRNA is regulated by Bcr-Abl in CML, and as sensitivity of LEF expression to imatinib or other therapeutic drugs targeting Bcr-Abl is reflective and/or prognostic of treatment outcome with imatinib and such other drugs, test systems and methods are contemplated in which sensitivity of LEF expression to imatinib in CML patients is assessed to provide guidance for treatment of the patients with imatinib.

Additionally, and in yet another aspect of the inventive subject matter, based on the inventors' discovery that LEF-1 is a target of Bcr-Abl (since production of LEF1 mRNA and LEF-1 protein are down-regulated by the Bcr-Abl inhibitor, imatinib), the inventors contemplate that LEF-1 is a target of Bcr-Abl signaling and may thus contribute to the increased survival and proliferation of myeloid progenitor populations. While not limiting to a particular theory or hypothesis, the inventors consequently contemplate that LEF-1 could play a role in the pathogenesis of CML (which would also be supported by the observation that the LEF-1 protein is capable of transforming cells, and that the LEF-1 protein is uniformly expressed in all primary CML samples tested).

Furthermore, the degree to which LEF-1 expression was inhibited by imatinib in vitro in cells from CML patients correlated with their clinical outcome. Remarkably, lack of inhibition was associated with the eventual development of clinical imatinib-resistance. Consequently, and viewed from a different perspective, it should be appreciated that LEF-1 expression may also play a role in conferring drug resistance in CML. It should be especially appreciated that of the known mechanisms associated with clinical imatinib-resistance, Abl kinase domain mutations account for the majority of cases. However, recent work has shown that in myeloid blast phase, the proportion of patients without kinase domain mutations to account for their imatinib-resistance may be as high as 41%, suggesting that alternative mechanisms of resistance become increasingly important in the later phases of disease. It should further be appreciated that disorders associated with IRES-regulated LEF-1 (and other transcription factors) may also be diagnosed or even treated by observing and/or interfering with cellular components that are functionally associated with LEF-1 expression.

For example, the inventors examined LEF1 expression in primary CML cells and cell lines (K562 and Ba/F3-Bcr-Abl) and showed that LEF-1 protein was detected in all patient derived cells. Treatment of these cells with the Bcr-Abl inhibitor imatinib mesylate (imatinib) inhibited LEF-1 expression in imatinib-sensitive cancers, but not in cancers that exhibited clinical resistance even though such cancers expressed imatinib-sensitive Bcr-Abl. For those cancers that were sensitive, inhibition of Bcr-Abl had a partial effect on LEF1 mRNA levels, and a significant effect on LEF-1 protein levels. LEF-1 protein was produced via two internal ribosome entry sites (IRES) in its 5′-UTR (untranslated region). IRES-driven translation of LEF-1 was highly sensitive to Bcr-Abl as treatment with imatinib reduced IRES activity 5 fold. Transfection of CML cells with dicistronic mRNAs suggests that Bcr-Abl stimulates LEF-1 protein production through steps in the nucleus and cytoplasm. It is therefore contemplated that, in addition to its strong effects on cap-dependent translation in CML, Bcr-Abl is an important regulator of alternative translation pathways.

Consequently, the inventors contemplate a combination test in which a patient sample is tested for catalytic activity of the kinase domain and tested for the amount of LEF-1. Among other test systems, it is generally preferred that the test for catalytic phosphorylation activity is based on a Western blot in which phosphorylated product is detected/quantified, and in which expression of the LEF-1 protein is detected/quantified via Western blot. Furthermore, and where alternative test methods for kinase activity determination are desired, it should be appreciated that all of the known methods are deemed suitable for use herein (see e.g., Anal Biochem. 2005 Dec. 1; 347(1):67-76; Cytometry A. 2004 November; 62(1):35-45; Blood. 2005 Feb. 15; 105(4): 1652-9). For example, kinase activities could be determined using chromogenic or fluorogenic substrates, antibody sandwich assays, mass spectroscopy of test fluids containing kinase substrates, etc. Similarly, with respect to detection and/or quantitative analysis of LEF-1, it should be recognized that all manners of quantification of a known protein are deemed suitable for use herein. For example, LEF-1 can be quantified via Western blot, LC-MS, etc. In a typical test, a first aliquot of patient cells is cultured and/or treated in the presence of imatinib, while a second aliquot of patient cells is cultured as control without treatment/exposure to imatinib. The cells are then assayed for activity of Bcr-Abl kinase and for expression level of LEF-1. Abrogation of kinase activity and LEF-1 expression in the presence of imatinib is indicative of a likely responder to imatinib treatment, while sustained kinase activity and sustained LEF-1 expression in the presence of imatinib is indicative of a likely non-responder to imatinib treatment. Where the kinase activity is reduced and LEF-1 expression is not reduced, it is likely that the patient is a poor responder, a non-responder, or will develop (or select for) imatinib resistance during treatment.

In a still further contemplated aspect, it should be noted that LEF-1 is associated with an IRES and frequently up-regulated in stress response and/or rapidly dividing cells, and especially that in neoplastic cells, two versions of LEF-1 have been described wherein one version may be associated with a cap-dependent translation while the other version acts in an ordinary and IRES characteristic manner. While not wishing to be bound by any theory or hypothesis, it is now contemplated that an imbalance in such transcripts may be associated with a dysregulation in growth in neoplastic cells. Therefore, IRES sequences may also be used as diagnostic landmarks to determine expression ratios of versions of genes functionally associated with the IRES sequence.

### Contemplated Therapeutic Uses

Since LEF1 is a necessary factor in CML, it is further contemplated that down-regulation of IRES activity might be a viable new target for CML. Remarkably, such opportunity may be present as the inventors discovered that Bcr-Abl up-regulates IRES activity. Imatinib treatment of K562s and a limited number of primary CML cells down-regulates LEF1 protein expression. Bcr-Abl is the target of Imatinib/Gleevec, and Bcr-Abl is known to regulate translation of cap-dependent mRNAs. LEF1 is translated by a cap-independent mechanism that is not very well defined, nevertheless our data and the results of other groups suggest that Bcr-Abl up-regulates cap-independent pathways of translation. Interestingly, many of the primary CML cancers that we assayed showed Imatinib insensitivity with regard to LEF1 expression. The inventors used Western analysis to determine whether Bcr-Abl protein was itself insensitive to Imatinib inhibition. The protein Crk1 is a direct protein kinase substrate for Bcr-Abl, and in all cases, Imatinib treatment, inhibited the phosphorylation of Crk1 by Bcr-Abl. This analysis showed directly that the Imatinib insensitivity of LEF1 expression was not due to drug resistant mutations of BcrAbl.

Based on these and other findings, the inventors contemplate that various options may be present in which LEF-1 inhibition can be achieved in a manner other and/or additional to Bcr-Abl inhibition. Such option was explored in the following experiment (data not shown):

A dicistronic vector carrying full-length LEF1 IRES was transiently transfected into K562 cells—a cell line with CML characteristics. The LEF1 IRES is highly active in K562 cells and Imatinib can inhibit this activity 3-4 fold. The inventors then set out to investigate whether interfering with another kinase (GSK3-beta) would influence IRES activity and would prevent Imatinib from repressing the IRES. To that end an expression vector was transiently co-transfected with a mutant, dominant, interfering version of GSK3-beta. This mutant version of GSK3-beta interferes with normal, wildtype GSK3-beta and its repressive activities on translation and Wnt signaling.

Remarkably, Imatinib treatment of transfected K562 cells inhibits the LEF1 IRES 3-fold (i.e. IRES activity is about 35% that of untreated cells). Co-transfection of a mutant GSK3-beta with the dicistronic vector had a positive effect on the LEF1 IRES, enhancing activity 160%+30% relative to untreated cells. Importantly Imatinib was much less able to inhibit the LEF1 IRES (activity was about 78% of untreated cells) when the mutant GSK3-beta was co-expressed, which strongly suggests that interference with GSK3-beta affects the efficacy of Imatinib inhibition of LEF1 IRES activity.

Based on these findings and other reports, it is contemplated that GSK3-beta (which is a ubiquitous and constitutively acting kinase) dampens translation by activating two related tumor suppressors (TSC1 and TSC2) that participate in translation pathways. GSK3-beta also inhibits the Wnt signal transduction cascade. Therefore, interfering with GSK3-beta by expressing a dominant negative version of itself, relieves the negative actions of GSK3 and thus allows for a more active translation pathway and active Wnt signaling (it was earlier demonstrated that late stage CML cancers have up-regulated Wnt signaling). Consequently, the inventors contemplate that late stage cancers may be insensitive to Imatinib in part because other pathways (i.e. GSK3-beta and/or the Wnt pathway) are redundant with Bcr-Abl actions. Therefore, new treatments may include those in which additional inhibitors are administered to reduce or even block activity if alternative pathways that provide Bcr-Abl actions or Bcr-Abl-like actions. Among other suitable targets, co-administration of Bcr-Abl inhibitors with Wnt pathway inhibitors and/or GSK3-beta activators are particularly preferred.

Experimental Data

### RNA Isolation and Northern Blot Analysis

Total RNA was isolated from K562 cells using TriZol reagent (Invitrogen) according to the manufacturer's instructions. Twenty-four hours prior to RNA isolation, K562 cells were treated with either DMSO, 2 μM imatinib, rapamycin (10 ng/mL) (Sigma-Aldrich), or both inhibitors in RPMI medium supplemented with 5% FBS at 37° C. Approximately 20 μg of total RNA from K562 cells was analyzed on a Northern blot for LEF1 expression, respectively. Northern blotting was carried out as described elsewhere and the LEF1 ORF (open reading frame) probe was generated as described elsewhere and radiolabeled with ³²P-dATP. The blot was stripped and rehybridized with a ³²P-dATP radioloabeled GAPDH loading control probe.

### Plasmids

The dual luciferase dicistronic plasmids, pRSTF and pRSTF-CVB3 are described in J Biol Chem, 279: 47419-47430, 2004. The dicistronic vector which contains 1.178 kb of the LEF1 5′UTR, pRSTF-LEF(1.2) is described in RNA, 11: 1385-1399, 2005. The LEF-1 open reading frame (ORF) construct used to express full length LEF-1 in Ba/F3 cells contains 1.2 kb of the 5′UTR, the full 1.2 kb ORF as well as the 1.2 kb 3′UTR, is described in RNA, 11: 1385-1399, 2005.

### Cell Lines and Cell Culture Conditions

The murine hematopoietic cell lines, Ba/F3-Bcr-Abl-WT and Bcr-Abl-T315I, as well K562 cells were previously described. The cell lines were cultured in RPMI 1640, 1X medium supplemented with 10% fetal bovine serum (FBS), 2 mM L-glutamine and antibiotics. Cells were maintained at 37° C. in a humidified atmosphere of 5% CO₂. All cell culture reagents were obtained from Invitrogen.

### Patient Samples and Cell Processing

Peripheral blood (PB) samples were obtained with appropriate consent and IRB approval from patients with CML at the University of California at Irvine. PB mononuclear cells (MNCs) were obtained by centrifugation through Ficoll-Hypaque, washed in PBS, and cryopreserved. To expand CML cells in vitro, cells were thawed and grown in media supplemented with 10% FBS and a growth factor cocktail consisting of 20 ng/ml granulocyte-macrophage colony stimulating factor, 20 ng/ml recombinant human interleukin 3, 20 ng/ml recombinant human stem cell factor, and 100 ng/ml granulocyte colony-stimulating factor. Following this, cells were incubated in liquid culture for 72-96 hrs, at 37° C. in a humidified atmosphere with 5% CO₂ in serum-free medium (SFM) (StemCell Technologies, Vancouver, BC, Canada) supplemented with growth factors (GF) at concentrations similar to that found in stroma-conditioned medium. All cytokines were from PeproTech, Rocky Hills, N.J., except for GM-CSF (sargramostim, Immunex, Seattle, Wash.) and G-CSF (filgrastim, Amgen, Thousand Oaks, Calif.).

### DNA Transient Transfections

For transfection of the dicistronic plasmids into Ba/F3 cells, approximately 10×10⁶ cells were seeded in 10 cm² plates and 10 μg of the reporter plasmid was transfected via a BTX 600 electroporator. Cells were allowed to recover for 2 hours in 37° C. and then treated with either DMSO (mock), 5 μm imatinib, 10 ng/mL rapamycin, or both inhibitors for 6 hours. Cell lysates were prepared for dual luciferase assays 8 hours post-transfection. K562 cells were treated with 2 μM imatinib for 22 hours at 37° C. Cell lysates were prepared for dual luciferase assays 24 hours post-transfection. Transfections were performed in duplicate and each experiment was carried out at least 3 times.

### Dual Luciferase Assays

Cell lysates were assayed for luciferase activities using the Dual Luciferase Reporter Assay System (Promega) according to the manufacturer's protocol. Assays were performed using a SIRIUS luminometer (Berthold Detection Systems). FLuc:RLuc ratios were calculated for each sample, and the average value is represented as fold elevation over background, which is the Firefly luciferase: Renilla luciferase ratio (FLuc:RLuc) measured from the vector in the absence of an insert.

### In Vitro Transcription

The capped dicistronic mRNAs were generated by in vitro transcription of the dicistronic (linearized with PMEI) plasmid using the T7-based Megascript in vitro transcription kit (Ambion) as per the manufacturer's protocol. The integrity of the transcripts was analyzed on a 1% agarose gel.

### RNA Transient Transfections

Approximately 6×10⁵ cells (K562) were seeded in 6-well plates and 2 μg of in vitro synthesized dicistronic mRNA was transfected per well using the Transmessenger transfection kit (Qiagen). A ratio of 1:6 (1 μg of RNA: 6 μL of transmessenger reagent) was used according to the manufacturer's instructions. Two hours post-transfection, the cells were treated with 2 μM imatinib for an additional 22 hours at 37° C. in RPMI media supplemented with 5% FBS. Cell lysates were then assayed for luciferase activities as described above. All transfections were performed in duplicate and carried out 3 times.

### Western Blot Analysis

Whole cell lysates from Jurkat, K562, and Ba/F3-Bcr-Abl-WT and Ba/F3-Bcr-Abl-T3151 cells (transfected with 10 μg of the LEF-1 expression plasmid) were separated by electrophoresis on 10% SDS-PAGE (polyacrylamide gels) and probed with the indicated antibodies. LEF-1N polyclonal rabbit antisera (detects all LEF/TCF proteins) was used at a 1:1000 dilution and anti-actin antisera (Santa Cruz Biotechnology) was used at a 1:500 dilution. Immunoreactive bands were visualized by enhanced chemiluminescence (Amersham Biosciences) after incubation with secondary antibody.

### Results

### LEF-1 Expression in Chronic Myeloid Leukemia

As it had been shown that LEF1 transcripts are upregulated in advanced CML, the inventors proceeded to examine whether LEF-1 protein is detectable in primary CML isolates, and whether expression is due to the actions of Bcr-Abl and/or mTOR, a central regulator of cap-dependent translation. Primary lysates from three patients with chronic phase CML and one patient in blast phase were examined for LEF-1 expression using Western blot analysis with a polyclonal antibody that detects all forms of LEF-1 protein. Prior to harvest, primary CML cells were treated for 24 hours with either 2 μM imatinib, 10 ng/ml rapamycin, or both to inhibit the Bcr-Abl and mTOR kinases respectively. Blots were also probed with CrkL antibody to confirm inhibition of Bcr-Abl kinase activity by imatinib, and re-probed with actin antibody for a loading control. Full length LEF-1 is frequently expressed as a cluster of polypeptides from 53-57 kDa due to alternative splicing in the middle and 3′ end of the locus.

FIG. 1 depicts LEF-1 expression in primary CML. Immunoblots showing LEF-1 expression in cell lysates from 3 patients (CML-6, -7, -14) in chronic phase (CP) CML, and one patient (04-1) in blast phase (BP). Cells were treated with DMSO (D), 2 μM imatinib (I), rapamycin (R, 10 ng/mL) or both (28) for 24 hours. A polyclonal LEF-1 antibody that detects all forms of LEF-1 protein was used to detect LEF-1. Blots were also probed with CrkL antibody to confirm inhibition of Bcr-Abl by imatinib, as well as an actin antibody for a loading control. The CrkL antibody detects both phosphorylated and nonphosphorylated protein. In all samples, the presence of imatinib inhibited formation of the lower mobility band, which corresponds to phosphorylated CrkL, indicating inhibition of Bcr-Abl.

As can be taken from FIG. 1, a major isoform of 55 kDa, and sometimes a minor 57 kDa isoform, is expressed in all four primary CML samples. Also, LEF-1 protein levels appear to be comparable between chronic phase and blast phase samples. Since LEF1 mRNA has been reported to be increased in blast phase cells compared to those from chronic phase, it is therefore likely that LEF-1 protein expression is primarily regulated by post-transcriptional mechanisms. In chronic phase CML cells, the inventors found that LEF-1 protein expression was dependent on Bcr-Abl kinase activity, although the degree of dependence ranged from almost complete dependence (CML-6) to partial dependence (CML-7 and CML-14). In contrast, in blast phase CML cells (CML-04-01), the inventors found that LEF-1 expression was completely independent of Bcr-Abl kinase activity.

In all cases, mTOR activity did not contribute significantly to LEF-1 expression. To determine if the variable sensitivity of LEF-1 expression to imatinib might correlate with clinical outcome, the inventors reviewed the clinical response of the patients to imatinib treatment. Interestingly, the inventors found that patient CML-6 had the best clinical response to imatinib, and was able to achieve a complete cytogenetic response (CCR) within 7 months of initiating therapy. LEF-1 expression was markedly inhibited by imatinib in this cancer. In contrast, patients with poor clinical outcomes had cancers where LEF-1 expression exhibited varying levels of insensitivity to imatinib. Patient CML-7 failed to achieve a CCR and went on to develop accelerated phase disease within two years of starting imatinib therapy, while patient CML-14 failed to even achieve a complete hematologic response. The patient whose cells exhibited no LEF-1-sensitivity to imatinib (CML-04-01) fared the worst, and died with imatinib-resistant myeloid blast phase disease within three months of providing the sample. These results demonstrate that LEF-1 protein is expressed in all stages of CML and that Bcr-Abl regulates LEF-1 expression in imatinib-sensitive CML.

### Effects of Imatinib on LEF-1 Protein Expression

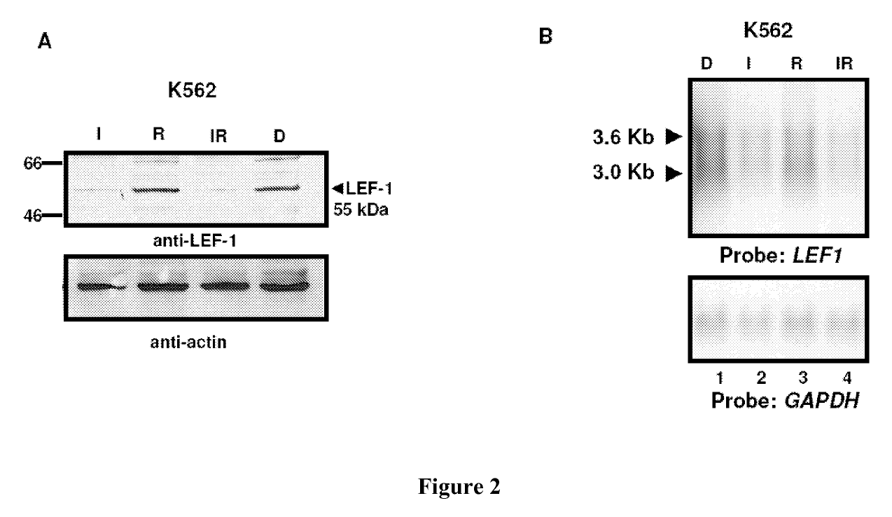

To examine LEF-1 expression in a CML cell line, the inventors used K562 cells for Western and Northern blot analysis, and the results are depicted in FIGS. 2A and 2B depicting effects of imatinib on LEF-1 expression. Panel A: Whole cell lysates from K562 cells treated with DMSO (D), imatinib (I, 2 uM) rapamycin (R, 10 ng/mL) or both (28) for 24 hours were analyzed for LEF-1 protein expression by Western blot with the indicated antibodies. Panel B: Northern blot analysis of total RNA isolated from K562 cells treated with inhibitors as described above. The three LEF1 mRNAs are indicated by arrows. A radiolabeled LEF-1 ORF cDNA probe was used to detect LEF1 mRNAs in the top panel. The blot was stripped and reprobed with GAPDH.

As can be seen from the figures, a single 55 kDa LEF-1 polypeptide was detected on Western blots. A higher molecular weight protein was also detected at low levels and this may correspond to SUMO-modified LEF-1 protein. Importantly, treatment of K562 cells with imatinib significantly down-regulated LEF-1 protein levels. As was observed for primary CML, rapamycin had no effect, and the two drugs together were as effective as imatinib alone. The inventors therefore conclude that K562, which is an imatinib-sensitive CML cell line, is a good model system to study LEF-1 regulation by Bcr-Abl, since it recapitulates the pattern of drug sensitivities seen with the imatinib-sensitive patient sample. Decreases in protein levels can be due to regulation at many different steps from transcription to translation. The LEF1 promoter has been previously defined and shown to be subject to regulation by signaling pathways, such as Wnt and TGF-alpha. Therefore, to examine the regulation of the Bcr-Abl pathway on LEF1, Northern blot analysis was performed using RNA isolated from K562 cells treated with imatinib for 24 hours. In addition, cells were treated with rapamycin or the combination of both drugs.

The Northern blot analysis using a LEF1 open reading frame probe detects two messages (FIG. 2B, arrows: 3.6 kb and 3.0 kb) with long 5′UTRs which are generated by two closely spaced promoters in the LEF1 gene. The 3.6 kb and 3.0 kb mRNAs produce full-length LEF-1 protein via internal initiation translation mechanisms and both are downregulated by imatinib (3 fold) while rapamycin causes an almost 2 fold decrease. The combined treatment reduces total LEF1 mRNA levels by almost 4 fold. However, hybridization of the same blot with a GAPDH radiolabeled cDNA probe shows that GAPDH levels are also reduced by imatinib treatment (1.4 fold; FIG. 2: lanes 3 and 5). After normalizing with GAPDH mRNA levels, the inventors determined that imatinib decreased LEF1 mRNA by 2 fold.

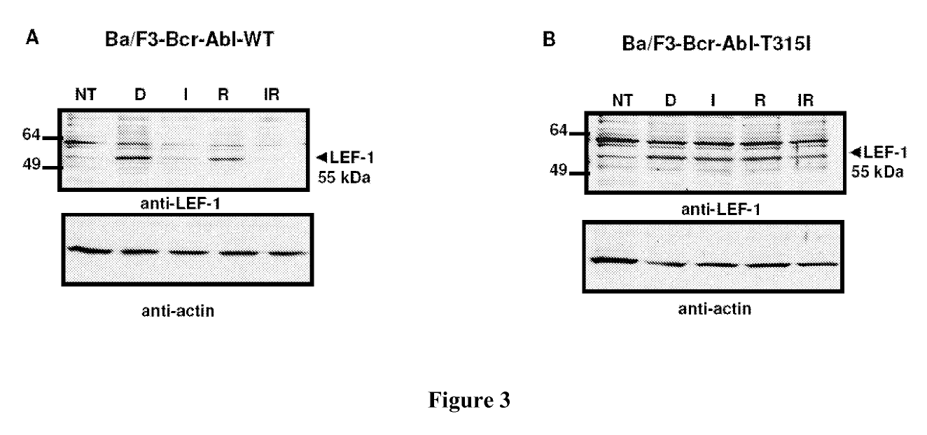

Another Bcr-Abl cell line was used to probe for regulation of LEF-1 to test whether production of LEF1 mRNA by a strong, heterologous viral promoter exhibited the same sensitivity to imatinib as shown in FIG. 3. Here, LEF-1 expression in Bcr-Abl expressing cells is indicated as follows. Panel A: Ba/F3-Bcr-Abl-WT and Panel B: imatinib-resistant T315I cells were transfected with a LEF-1 expression plasmid containing full length 5′ and 3′ UTRs; NT=nontransfected control. Cells were reacted with inhibitors for 6 hours and Western blot analysis was performed with the indicated antibodies. Ba/F3-Bcr-Abl-WT and Ba/F3-Bcr-Abl-T315I cells are pre-B lymphocyte cell lines modified to express Bcr-Abl. Ba/F3-Bcr-Abl-WT cells stably express the imatinib-sensitive ‘wild-type’ Bcr-Abl while the Ba/F3-Bcr-Abl-T315I cells stably express an imatinib-resistant Bcr-Abl mutant. The inventors transiently transfected these cells with a LEF-1 expression plasmid driven by the highly active cytomegalovirus LTR promoter. This expression plasmid produces authentic, full-length LEF1 mRNA identical in sequence to the 3.6 kb mRNA detected on Northern blots.

FIG. 3A shows that treatment with imatinib in Ba/F3-Bcr-Abl-WT cells down-regulates LEF-1 protein levels to very low levels. Rapamycin treatment of the cells had a slight effect on LEF-1 and the combined drug treatment had a slightly greater effect than imatinib alone, lowering LEF-1 protein to undetectable levels. This striking regulation was not observed in the Ba/F3-Bcr-Abl-T315I cells which express the imatinib-insensitive Bcr-Abl mutant. Neither imatinib and/or rapamycin treatments had any effect on LEF-1 as can be seen from FIG. 3B.

This data demonstrates that Bcr-Abl specifically and strongly regulates LEF-1 expression. While some regulation may occur at the level of transcription, the fact that LEF-1 protein was completely shut down in the Ba/F3-Bcr-Abl-WT cells transfected with a heterologous expression plasmid suggests that much of Bcr-Abl's effect could be posttranscriptional. Indeed, the Northern blot analysis does not distinguish between an effect at the level of transcription or an effect on mRNA stability. Taken together, these data suggest that Bcr-Abl might regulate LEF-1 expression at post-transcriptional steps.

In addition to widespread effects on patterns of gene expression, including the transcription of protein biosynthesis genes, Bcr-Abl also directly upregulates cap-dependent translation. Thus, in the chronic phase and later stages, there is a notable elevation in protein production and metabolism. However, LEF-1 protein is not produced by cap-dependent translation. Instead, LEF-1 is produced via the action of two IRES modules in its long 5′-UTR. Bcr-Abl effects on this alternate mode of translation have not been investigated although positive effects in the IRES-containing c-Myc mRNA have recently been reported.

### Effects of Imatinib on Cap-Independent Translation

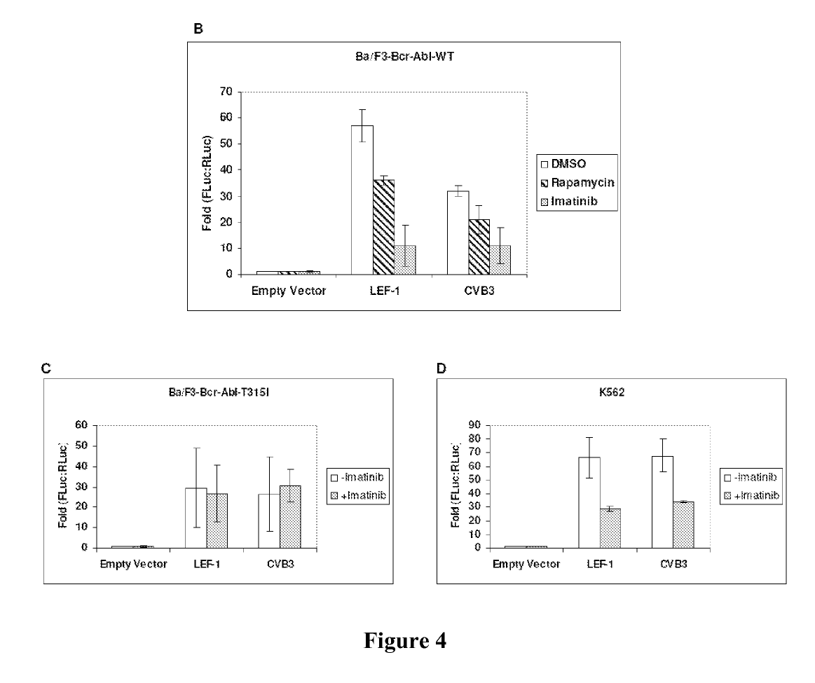

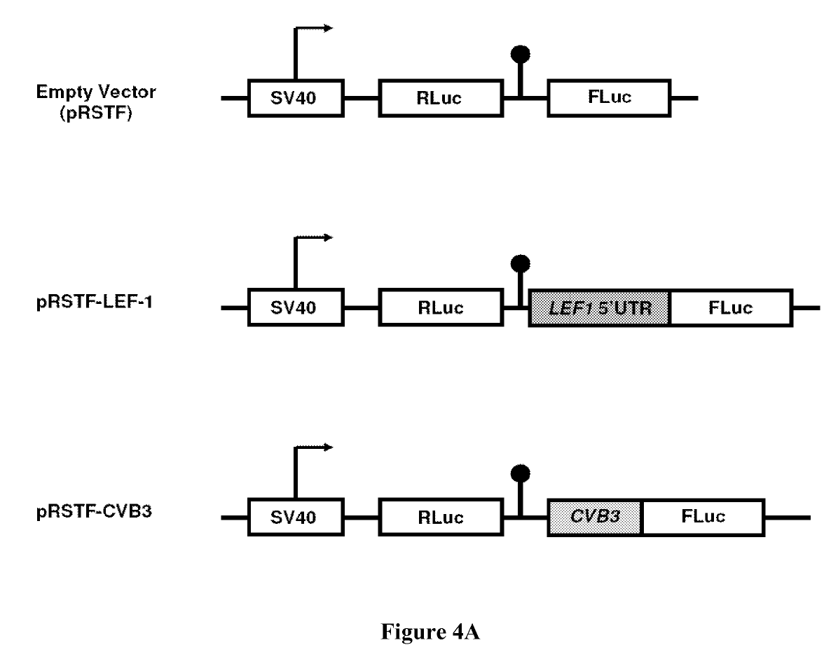

To better distinguish between transcription and translation as well as to examine the effect imatinib may have on LEF-1 IRES activity and subsequent protein production, transient DNA transfections were carried out using dicistronic plasmids as depicted in FIG. 4A in which RLuc=Renilla luciferase, FLuc=Firefly luciferase. A stem loop sequence (depicted by a bold line and circle) ensures separate translation of the cap-dependent RLuc ORF from the IRES-driven downstream cistron, FLuc. The effects of imatinib, rapamycin, or both on LEF-1 IRES activity is shown in FIG. 4B for Ba/F3-Bcr-Abl-WT cells, in FIG. 4C for Ba/F3-Bcr-Abl-T315I cells, and in FIG. 4D for K562 cells transfected with the dicistronic constructs. IRES activity is represented as the ratio of FLuc:RLuc and the y-axis on the graphs represents the fold elevation above the empty vector set equal to one.

The dicistronic system has been widely used to demonstrate cap-independent initiation of translation. The dicistronic vector depicted in FIG. 4A contains 2 cistrons separated by an intercistronic region that also contains a hairpin structure. The upstream cistron codes for Renilla Luciferase (RLuc) while the downstream cistron encodes Firefly Luciferase (FLuc). The SV40 promoter drives transcription of the dicistronic mRNA, and translation of RLuc relies on cap-dependent ribosome-scanning mechanisms while translation of the downstream cistron relies on internal ribosome entry. Hence, the dicistronic vector with no insert (pRSTF) will only produce Renilla Luciferase because the hairpin structure inserted between the two cistrons prohibits ribosomal read-through. On the other hand, when a sequence known to contain an internal ribosome entry site (IRES) is inserted in the intercistronic region, Renilla and Firefly Luciferase proteins are produced albeit by different translation mechanisms. The inventors previously used this system to demonstrate that translation of LEF-1 is mediated via two IRES modules located in the 5′UTR of LEF1 mRNA. A dicistronic construct that contains the full length 1.2 kb LEF1 5′UTR and IRES, the empty vector with no insert (pRSTF), and the viral IRES of the well characterized coxsackievirus B3 (CVB3) were analyzed for comparison. IRES elements of viral origin have been very well characterized and are highly efficient while those of cellular mRNAs are not. In addition, cellular IRESs appear to be regulated via different factors and or conditions. Therefore, the inventors included CVB3 in their studies to ensure that the dicistronic reporter plasmid is expressed in the cell lines and to also examine whether the effects the inventors observe in their experiments are specific to LEF-1. The dicistronic plasmids were transiently transfected into Ba/F3 as well as into K562 cells. The dicistronic system uncouples transcription and translation because the ratio of proteins produced, in this case RLuc and FLuc, are generated from a single mRNA and can be compared. IRES activity is represented as the fold change in the ratio of FLuc:RLuc compared to the ratio obtained from the dicistronic vector with no insert. Thus a functional LEF1 IRES increases translation of FLuc and changes the FLuc:RLuc ratio.

FIG. 4B demonstrates the effects of imatinib, rapamycin or both on LEF1 IRES activity in Ba/F3-Bcr-Abl-WT cells. Cells were harvested 8 hours post-transfection and dual luciferase assays were carried out to examine LEF-1 protein synthesis via IRES12 mediated translation. The LEF1 IRES is highly active in these cells, causing a 55 fold elevation in the FLuc:RLuc ratio. The CVB3 IRES also functions in these cells, but is not as active as LEF1 (33 fold elevation). Treatment with imatinib caused a 5 fold decrease in IRES activity for LEF1, while CVB3 IRES activity was affected about 3 fold. It is possible that Bcr-Abl affects some of the same factors employed by both viral and cellular IRES elements, but these data show that the LEF1 IRES is more sensitive. Treatment with rapamycin had a mild effect on LEF-1 (38% decrease) and a similar change in activity was observed with CVB3 (33% decrease). With regard to cap-dependent translation, RLuc activity was decreased by an average of 5-fold in the presence of imatinib and 2 fold with rapamycin treatment. These results are consistent with previous studies showing important effects of Bcr-Abl on cap-dependent translation. The inventors have shown that the LEF-1 IRES is independent of a 5′-cap structure since transfection of capped and uncapped dicistronic mRNAs yields equivalent levels of protein from the downstream cistron. In addition, suppression of cap-dependent translation via co-expression of a viral protease did not affect LEF-1 IRES activity. Therefore, the equivalent magnitude of imatinib effects on cap-dependent and LEF-1 IRES mediated translation is due to modulation of two distinct pathways. As a control, the effects of imatinib on LEF-1 IRES activity were examined in the imatinib-resistant Ba/F3-Bcr-Abl-T315I cells as can be seen in FIG. 4D. The results clearly show that treatment with imatinib had no effect on LEF-1 or CVB3 in these cells. In addition, a 2.3 fold decrease in LEF-1 IRES activity resulted from a 24 hour treatment with 2 μM of imatinib in K562 cells (see FIG. 4B). This less dramatic effect may be due to the fact that these cells were treated with a lower concentration of imatinib whereas the Ba/F3-Bcr-Abl-expressing cells were treated with a higher dose of imatinib for a shorter period. The inventors conclude that Bcr-Abl regulates LEF-1 expression primarily at the level of IRES-mediated translation.

### Imatinib Down-Regulates LEF-1 Via Steps in the Nucleus as Well as the Cytoplasm

To determine whether Bcr-Abl regulation is compartmentalized and further examine whether it functions post-transcriptionally, an RNA transient transfection was carried out in K562 cells. In an RNA transfection, in vitro synthesized dicistronic mRNA is introduced directly into the cytoplasm and does not cycle through the nucleus; therefore, any nuclear factors involved in post-transcriptional regulation of the mRNA are largely excluded. In these experiments the inventors again utilized CVB3 as a control for regulation in subcellular compartments since it predominantly depends on cytoplasmic machinery for translation. To faithfully mimic mRNAs, dicistronic transcripts were synthesized in vitro in the presence of a cap analog and the capped RNAs were transfected into K562 cells so that the effects of imatinib on cap-dependent and cap-independent translation could be compared. The cells were treated with 2 μM imatinib for 24 hours.

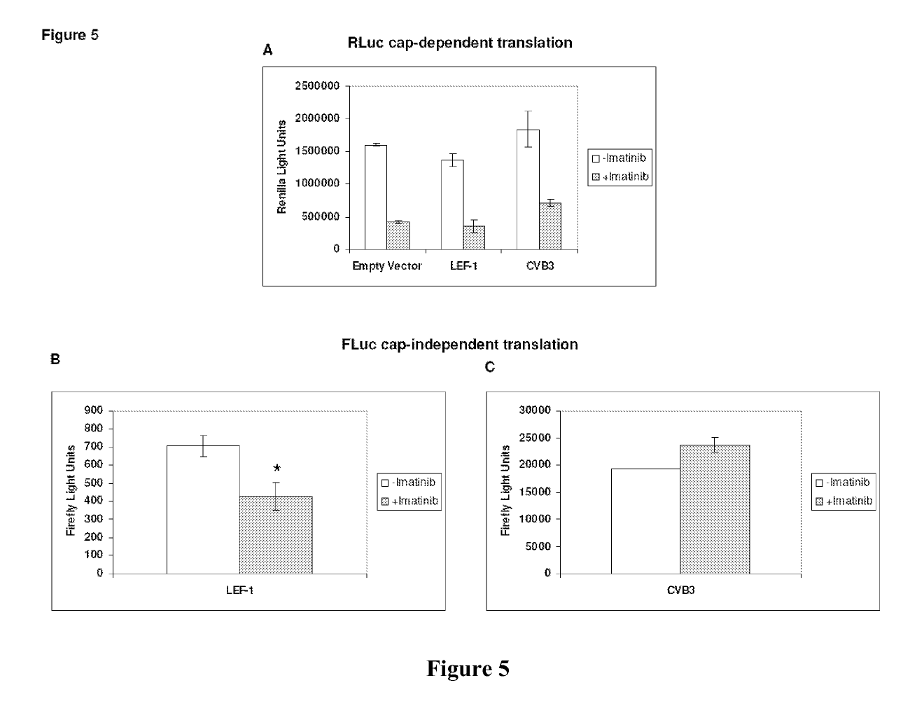

Imatinib-sensitive steps also occur in the cytoplasm as can be taken from FIGS. 5A-5C. Here, capped dicistronic mRNAs were synthesized in vitro and transiently transfected into K562 cells +/−2 μM imatinib (24 hour treatment) and cell lysates were assayed for luciferase activities. FIG. 5A illustrates Renilla luciferase activity generated by the dicistronic mRNAs which represents capdependent translation in the absence (open bars) or presence (shaded bars) of 2 μM imatinib. FIG. 5B illustrates firefly luciferase activity generated by the LEF-1 (* p<0.001), and FIG. 5C illustrates CVB3 IRES+/−2 imatinib. The results for LEF-1 and CVB3 had to be plotted on separate graphs due to the significantly higher luciferase activity produced by CVB3. FIG. 5A shows that the transfected RNA was a very good substrate for translation in the cytoplasm of K562 cells. Cap-dependent translation of the Rluc cistron produced high levels of protein, and as predicted from the known effects of Bcr-Abl on cap-dependent translation, imatinib inhibited RLuc translation by an average of 3.4 fold (imatinib reduced RLuc translation an average of 2.6 fold in the DNA transfection of K562 cells). In contrast, the LEF-1 IRES was much weaker when transfected as a naked RNA template as can be taken from FIG. 5B. The inventors have reported similar observations in COS-1 cells and conclude that the LEF-1 IRES requires resident time in the nucleus to be recognized by factors and complexes that combine with the RNA to create a highly active IRES. Nevertheless, imatinib reduced the activity of the LEF-1 IRES in the cytoplasm by 45% (p<0.001). Finally, the CVB3 IRES was much more active than the LEF-1 IRES but was insensitive to the effects of imatinib as shown in FIG. 5C.

The CVB3 IRES is of viral origin and is significantly more active than the LEF-1 IRES (interestingly, this is not the case in the DNA transfection). The data from the DNA and RNA transfections highlight differences in the mechanisms by which these two IRES function. In the DNA transfection, CVB3 is affected via imatinib similar to LEF-1. However, the results from the RNA transfection are more representative for this IRES because it reflects events occurring only in the cytoplasm and this virus does not cycle through the nucleus for translation. The stronger effect of imatinib on LEF-1 in the DNA transfection versus the RNA transfection suggests that the predominant Bcr-Abl regulated step derives from a nuclear event or protein(s). This step, which remains undefined, nevertheless differs in important ways from Bcr-Abl regulation of cap-dependent translation (RLuc) where the dominant effect is known to be on proteins and pathways in the cytoplasm. Clearly there are two pathways that drive protein production and Bcr-Abl regulates both of them via different mechanisms.

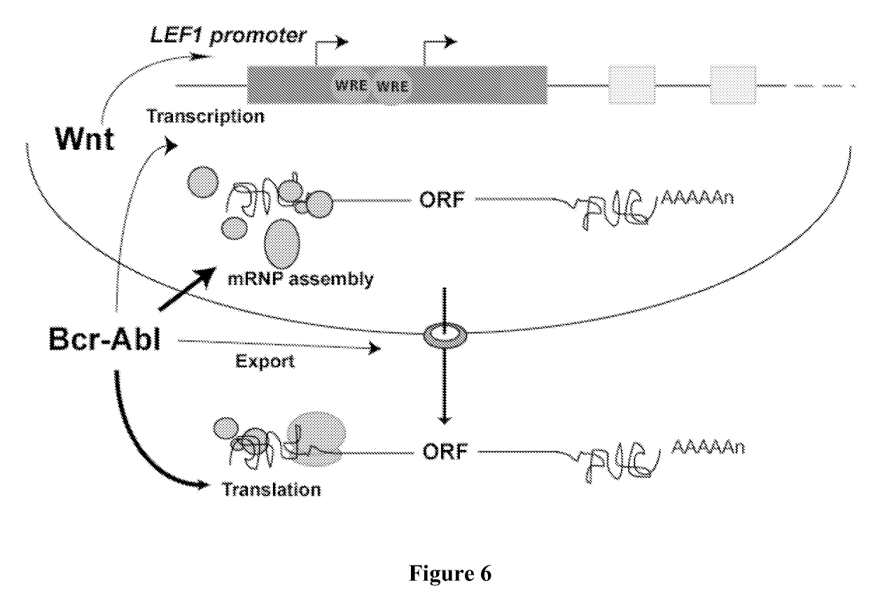

The inventors thus conclude that Bcr-Abl regulates cap-independent translation through novel steps in the nucleus and the cytoplasm. An exemplary model for such processing is schematically depicted in FIG. 6 in which the inventors contemplate that activated Wnt Signaling in late stages of CML enhances transcription of Wnt target genes, such as LEF1. The elevated LEF1 transcripts may then be regulated by Bcr-Abl at multiple steps; Bcr-Abl may modestly increase transcription of LEF1 in addition to up-regulating expression of RNA binding proteins that are involved in either nuclear splicing/export, and or IRES-mediated translation. This combined upregulation in transcription and translation results in increased protein synthesis of LEF-1 in CML.

Thus, specific embodiments, compositions, and methods relating to mammalian internal ribosome entry sites have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

What is claimed is:

1. A method of diagnosing a patient suffering from leukemia, comprising:
collecting leukocytes from the patient and measuring
(a) a first expression level of LEF-1 protein in the leukocytes prior to administration of a drug, and
(b) a second expression level of the LEF-1 protein in the leukocytes after administration of the drug;
comparing the first and second expression levels, optionally against an expression level of LEF-1 in leukocytes of a healthy person, to obtain a test result; and
determining a treatment option using the test result.

2. The method of claim 1 wherein measuring the first and second expression levels is performed using a labeled antibody having specificity towards LEF-1 protein.

3. The method of claim 2 wherein measuring the first and second expression levels is performed using a Western blot.

4. The method of claim 1 further comprising the steps of (a) measuring kinase activity of Bcr-Abl in the leukocytes prior to administration of the drug, and (b) measuring kinase activity of Bcr-Abl in the leukocytes after administration of the drug to obtain a second test result, and further using the second test result in the step of determining the treatment option.

5. The method of claim 4 wherein the step of measuring the kinase activity is performed using a fluorogenic or chromogenic substrate, optionally in Western blot format.

6. The method of claim 1 wherein the drug is a Bcr-Abl kinase inhibitor.

7. The method of claim 6 wherein the Bcr-Abl kinase inhibitor is selected from the group consisting of Dasatinib, Imatinib, and Nilotinib.

8. The method of claim 1 wherein the leukemia is chronic myeloid leukemia.

9. The method of claim 1 wherein the treatment option comprises administration of a Bcr-Abl inhibitor when the test result indicates that the second expression level is reduced as compared to the first expression level and optionally the expression level of LEF-1 in leukocytes of a healthy person.

10. The method of claim 1 wherein the treatment option comprises switching the patient to a different Bcr-Abl inhibitor when the test result indicates that the second expression level is not reduced as compared to the first expression level and optionally the expression level of LEF-1 in leukocytes of a healthy person.

11. An isolated IRES sequence, optionally upstream and/or downstream of an open reading frame coding for an expression product, wherein the IRES has a sequence that allows (a) hybridization under stringent conditions to the IRES of SEQ ID NO:1, and (b) increased expression of a reference peptide in an amount of at least 5 times relative to expression of the reference peptide under control of a mammalian Kv1.4 IRES in the same expression host.

12. The isolated IRES sequence of claim 11 wherein the IRES sequence has at least one of 70% sequence homology and 60% sequence length of the IRES of SEQ ID NO:1, and wherein the IRES sequence provides an at least 10 times increased expression relative to the reference peptide.

13. The isolated IRES sequence of claim 11 wherein the IRES sequence has at least one of 80% sequence homology and 70% sequence length of the IRES of SEQ ID NO:1, and wherein the IRES sequence provides an at least 20 times increased expression relative to the reference peptide.

13. The isolated IRES sequence of claim 11 wherein the IRES sequence has at least one of 95% sequence homology and 90% sequence length of the IRES of SEQ ID NO:1, and wherein the IRES sequence provides an at least 60 times increased expression relative to the reference peptide.

14. The IRES sequence of claim 11, wherein the IRES sequence is a sequence according to SEQ ID NO:1.

15. The IRES sequence of claim 11, comprising distinct upstream and downstream ORFs for expression in the host cell.

16. The IRES sequence of claim 11, wherein at least one of the upstream and downstream ORFs encodes a luciferase gene.

17. The IRES sequence of claim 14, further comprising sequences effective to allow at least one of integration of the sequence into a host genome and replication in the host cell.

18. The IRES sequence of claim 14, further comprising a selectable marker that allows selection for the sequence in the host cell.

19. A cell transfected with the IRES sequence of claim 11.

20. The cell of claim 19 wherein the cell is a leukocyte.
